(19) United States
(12) Patent Application Publication
(10) Pub. No.: US 2014/0058069 A1
(43) Pub. Date: Feb. 27, 2014

(54) PARTICLES AND OTHER SUBSTRATES USEFUL IN PROTEIN PURIFICATION AND OTHER APPLICATIONS

(71) Applicants: Boaz Mizrahi, Brookline, MA (US); Daniel S. Kohane, Newton, MA (US)

(72) Inventors: Boaz Mizrahi, Brookline, MA (US); Daniel S. Kohane, Newton, MA (US)

(73) Assignees: MASSACHUSETTS INSTITUTE OF TECHNOLOGY, CAMBRIDGE, MA (US); CHILDREN'S MEDICAL CENTER CORPORATION, BOSTON, MA (US)

(21) Appl. No.: 14/000,898

(22) PCT Filed: Feb. 22, 2012
(86) PCT No.: PCT/US12/26008

§ 371 Date: Oct. 29, 2013

Related U.S. Application Data
(60) Provisional application No. 61/445,942, filed on Feb. 23, 2011.

(51) Int. Cl. C07K 1/22; C08F 220/60; C07K 14/435

(52) U.S. Cl. CPC C07K 1/22; C07K 14/435; C08F 220/60
USPC 530/400; 530/413; 526/304; 525/328.3; 428/402

(57) ABSTRACT

The present invention generally relates to particles, including microgel particles, for purifying proteins and other species. In one aspect, the particles comprise a metal-chelating moiety, which may be distributed substantially evenly throughout the particle in certain embodiments. In some cases, the particles may be porous, and in some embodiments, the particles may be made sufficiently small, for example, in order to form a microgel containing the particles. Such particles may be useful, for example, in binding metal ions (for example, nickel ions) using the metal-chelating moieties. In some embodiments, such particles may also be used to bind certain analytes (for example, proteins) containing tags which attract metal ions, for example, histidine tags. Accordingly, in certain embodiments, the particles may be used for binding or trapping proteins. In some cases, this process is reversible; for example, upon exposure of the particles to a histidine competitor, proteins or other analytes containing the histidine tags may be released form the particles. Other aspects of the invention are generally directed to methods of using such particles, methods of forming such particles, kits including such particles, or the like.

### RELATED APPLICATIONS

This application claims the benefit of U.S. Provisional Patent Application Ser. No. 61/445,942, filed Feb. 23, 2011, entitled “Particles and Other Substrates Useful in Protein Purification and Other Applications,” by Mizrahi, et al., incorporated herein by reference.

### GOVERNMENT FUNDING

Research leading to various aspects of the present invention were sponsored, at least in part, by the NIH, Grant No. GM073626. The U.S. Government has certain rights in the invention.

### FIELD OF INVENTION

The present invention generally relates to systems and methods for purifying proteins and other species. In some embodiments, particles such as microgel particles are used for purification.

### BACKGROUND

Immobilized metal affinity chromatography (IMAC) is a frequently used method for the separation and purification of histidine-tagged (His-tagged) proteins. In this technique, the high affinity of metal ions such as nickel or cobalt to a tag sequence on the protein of interest creates strong yet reversible binding. One limitation of current systems is their inefficiency in purifying many recombinant proteins, particularly when present in their native state or in low concentrations in the cell lysate. Performance deficiencies may be caused, in part, by the clogging or destruction of matrix micropores by undissolved salts and other compounds during particle synthesis, limiting the surface area accessible for binding. Low surface metal density can also impair efficiency. Accordingly, improvements in the separation and purification of histidine-tagged proteins and other analytes are still needed.

### SUMMARY OF THE INVENTION

The present invention generally relates to systems and methods for purifying proteins and other species. In some embodiments, particles such as microgel particles are used for purification. The subject matter of the present invention involves, in some cases, interrelated products, alternative solutions to a particular problem, and/or a plurality of different uses of one or more systems and/or articles.

In one aspect, the present invention is generally directed to a composition. In one set of embodiments, the composition includes a particle formed from polymer. In some instances, the polymer comprises a metal-chelating moiety. In certain cases, the metal-chelating moiety is distributed substantially evenly throughout the particle.

In another aspect, the present invention is generally directed to a method of releasing a histidine-tagged analyte from a particle. The method, according to one set of embodiments, includes an act of exposing a particle suspected of being exposed to a histidine-labeled analyte to a histidine competitor. In some embodiments, the particle contains a metal ion distributed substantially evenly throughout the particle.

The present invention, in yet another aspect, is generally directed to a method of releasing a histidine-tagged analyte from a particle. In certain embodiments, the method includes an act of exposing, to a histidine competitor, a particle containing a histidine-labeled analyte distributed substantially evenly throughout the particle.

According to still another aspect, the present invention is generally directed to a method including acts of exposing a solution comprising a metal-chelating moiety and acrylamide to a liquid that is immiscible with the solution to form droplets of the solution contained within the liquid, polymerizing the metal-chelating moiety with the acrylamide to form polymeric particles, and separating the polymeric particles from the liquid.

In another aspect, the present invention encompasses methods of making one or more of the embodiments described herein, for example, making particles such as microgel particles. In still another aspect, the present invention encompasses methods of using one or more of the embodiments described herein, for example, using particles such as microgel particles.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIGS. 1A-1D illustrate various emission intensities of certain particles, illustrating protein immobilization in accordance with certain embodiments of the invention;

FIGS. 2A-2D illustrate various SEM micrographs of particles in accordance with various embodiments of the invention;

FIG. 3 shows a comparison of certain microgel particles and commercially available beads in purifying proteins, in yet another embodiment of the invention;

FIG. 4 illustrates an example scheme for preparing microgel particles;

FIGS. 5A-5B show various confocal micrographs of certain microgel particles and commercially available beads, in still another embodiment of the invention; and

FIG. 6 shows ¹H NMR spectra of certain particles of the invention.

### DETAILED DESCRIPTION

The present invention generally relates to systems and methods for purifying proteins and other species. In some embodiments, particles such as microgel particles are used for purification. Certain aspects of the present invention are generally directed to particles or other substrates for uses in purifying proteins and other species. In some cases, the substrates may be formed from or include polymers. In one aspect, the substrate comprises a metal-chelating moiety, which may be distributed substantially evenly throughout the substrate in certain embodiments. In some cases, the substrate may be porous. The substrate, in one set of embodiments, may be substantially spherical and/or may be sufficiently small, for example, in order to form a microgel containing particles. The substrate may also include metal ions (for example, nickel ions or cobalt ions) that associate with the metal-chelating moieties. In some embodiments, the substrate may be used to bind certain analytes (for example, proteins) containing tags, such as histidine tags, which are attracted to metal ions. Accordingly, the substrates may be used for binding or trapping analytes such as proteins. In some cases, this process is reversible; for example, upon exposure of the substrate to a histidine competitor, proteins or other analytes containing the histidine tags may be released form the substrates. Other aspects of the invention are generally directed to methods of using such substrates, methods of forming such substrates, kits including such substrates, or the like.

Certain aspects of the present invention are generally directed to substrates, such as particles, for purifying analytes such as proteins. In one set of embodiments, the substrate comprises a metal-chelating moiety (e.g., nitrilotriacetic acid or a nitrilotriacetic acid derivative), which can bind a metal ion (e.g., Ni²⁺ or Co²⁺) that may be present on or in the substrate. A suitable analyte, such as a protein, may include a tag (e.g., a histidine tag), which is attracted and able to bind the metal ion. Thus, when the substrate is exposed to a sample suspected of containing a suitably tagged analyte, the tag may become associated with the metal ions present within the substrate. In some cases, this association is not permanent, and may be reversed, e.g., upon exposure to a suitable competitor (e.g., imidazole or histidine) to cause the analyte to dissociate from the metal ions, and thus to dissociate from the substrate. The analyte may then be collected, e.g., as a purified product, or for further use.

The substrate may have any suitable shape. For example, the substrate may be formed as particles, as a planar substrate, or the like. In one set of embodiments, the substrate is polymeric. For example, the substrate may include a polymer such as poly(acrylamide), e.g., formed through the polymerization of acrylamide and a suitable metal-chelating moiety, as discussed below. For instance, acrylamide may be polymerized to form poly(acrylamide) upon exposure to ammonium persulfate, methylenebisacrylamide, and/or N,N,N′,N′-tetramethylethylendiamine (“TEMED”). In some cases, the polymerization may occur within an emulsion, e.g., to form particles. For example, an emulsion may be formed where monomers are present within discrete droplets (e.g., in an aqueous environment) contained within a continuous phase (e.g., an organic or “oil” environment), and polymerization induced within the discrete droplets to form polymeric particles.

Other examples of suitable polymers that can be used in the substrate include, but are not limited to, poly(styrene), poly(propylene), poly(ethylene), agarose, and the like, e.g., in addition to and/or instead of poly(acrylamide). Still other examples include polyethylene, polystyrene, silicone, polyfluoroethylene, polyacrylic acid, a polyamide (e.g., nylon), polycarbonate, polysulfone, polyurethane, polybutadiene, polybutylene, polyethersulfone, polyetherimide, polyphenylene oxide, polymethylpentene, polyvinylchloride, polyvinylidene chloride, polyphthalamide, polyphenylene sulfide, polyester, polyetheretherketone, polyimide, polymethylmethacylate and/or polypropylene. Polymeric particles or other substrates formed using these polymers may be formed using techniques known to those of ordinary skill in the art.

In one set of embodiments, the substrate may be positively or negatively charged, e.g., to facilitate separation of proteins, or other analytes. For example, an analyte may be positively charged and a negatively charged substrate may facilitate attraction of the analyte. For instance, a protein may be positively charged due to residues such as glutamine or asparagine on the protein, which may be attracted to negatively charged particles or other substrates. As another example, an analyte may be negatively charged, and a positively charged particle may facilitate attraction of the analyte. For instance, a nucleic acid such as DNA or RNA may be negatively charged, and the nucleic acid may be attracted to positively charged particles or other substrates. In one set of embodiments, an acrylic acid or other monomer producing negatively charged residues may be incorporated into the polymer or otherwise added to the substrate to impart a negative charge on the substrate. In another set of embodiments, a monomer producing positively charged residues (e.g., ethylenimine), may be used to impart a positive charge on a substrate, e.g., via incorporation into the polymer or other addition to the substrate.

As mentioned, the substrate may take the form of one or more particles. In some cases, the particles may include microparticles and/or nanoparticles. A “microparticle” is a particle having an average diameter on the order of micrometers (i.e., between about 1 micrometer and about 1 mm), while a “nanoparticle” is a particle having an average diameter on the order of nanometers (i.e., between about 1 nm and about 1 micrometer. As additional examples, the particles may have an average diameter of less than about 5 mm or 2 mm, or less than about 1 mm, or less than about 500 micrometers, less than about 200 micrometers, less than about 100 micrometers, less than about 80 micrometers, less than about 60 micrometers, less than about 50 micrometers, less than about 40 micrometers, less than about 30 micrometers, less than about 25 micrometers, less than about 10 micrometers, less than about 3 micrometers, less than about 1 micrometer, less than about 300 nm, less than about 100 nm, less than about 30 nm, or less than about 10 nm. In some embodiments, the particle may have an average diameter of at least about 1 micrometer or at least about 10 micrometers. Also, the particles may be spherical or non-spherical. If the particle is non-spherical, the particle may have a shape of, for instance, an ellipsoid, a cube, a fiber, a tube, a rod, or an irregular shape. The average diameter of a non-spherical particle is the diameter of a perfect sphere having the same volume as the non-spherical particle.

In certain embodiments, the substrate may be a gel. Non-limiting examples of gels include poly(acrylamide) gel or agarose gel, or other gel materials such as those describe herein. For example, if the substrate is a particle, then the substrate may take the form of microgel particles or gel microparticles. In some embodiments, the gel particles may be collected together to form a gel material or a “microgel.” A gel typically is relatively solid or jelly-like, and may include a cross-linked polymer to form its structure. In some cases, the gel may be a hydrogel, e.g., a gel that contains water.

The substrate may be porous, in certain embodiments of the invention. In some embodiments, the substrate may have a relatively high surface area, for example, having an average surface area of at least about 5 m²/g, at least about 7 m²/g, or at least about 10 m²/g. In some embodiments, the substrate may have an average pore volume of at least about 0.005 cm³/g, at least about 0.01 cm³/g, or at least about 0.02 cm³/g. In other embodiments, the substrate may have an average pore width of at least about 5 nm, at least about 7 nm, or at least about 8 nm. Such porosities and dimensions may be determined using techniques known to those of ordinary skill in the art, for example, TEM, SEM, BET, or the like. The porosity may be created, for example, due to the nature of the polymer (e.g., certain gel polymers such as those described herein typically will form relatively porous structures), or the porosity may be induced by adding another material to the substrate that can be removed, thereby creating porosity within the substrate. For example, salts or other species that can be subsequently dissolved may be incorporated within the substrate.

The substrate may also comprise a metal-chelating moiety, for example, EDTA (ethylenediaminetetraacetic acid) or NTA (nitrilotriacetic acid), or derivatives thereof, to which metal ions, including divalent metal ions, are able to bind. Other non-limiting examples of metal-chelating moieties include various polyamino carboxylic acid such as Fura-2, iminodiacetic acid, diethylene triamine pentaacetic acid (DTPA), ethylene glycol tetraacetic acid (EGTA), 1,2-bis(o-aminophenoxy)ethane-N,N,N′,N′-tetraacetic acid (BAPTA), 1,4,7,10-tetraazacyclododecane-1,4,7,10-tetraacetic acid (DOTA), or the like, and derivatives thereof. The metal-chelating moiety may be one which is able to bind or complex with metal ions, such as divalent metal ions. Non-limiting examples of ions that can be chelating by metal-chelating moieties include example nickel, cobalt, calcium, iron, or the like. As a specific non-limiting example, a metal-chelating moiety may bind to nickel ions, so that a substrate containing the metal-chelating moiety may also contain nickel ions distributed within the substrate.

In some embodiments, the metal-chelating moiety may be incorporated into the polymeric structure of a substrate. The metal-chelating moiety may be present as a monomer as various monomers are polymerized and/or cross-linked to form a polymeric substrate, e.g., forming a copolymer or an interpenetrating network of polymers. For example, the metal-chelating moiety, may be incorporated in a polymer as a monomer such that when the polymer is formed, one of the monomers or residues within the polymer is the metal-chelating moiety. As a specific non-limiting example, an NTA derivative such as 2,2045-acrylamido-1-carboxypentylazanediyl)diacetic acid may be used, which forms NTA residues when incorporated within a polymer.

In some cases, the metal-chelating moiety may be distributed substantially evenly throughout the substrate. For example, the concentration of the metal-chelating moiety on the surface of the substrate and in the bulk or the center of the substrate may be substantially the same. For instance, the difference in concentration of the metal-chelating moiety between the surface of the substrate and the bulk or center of the substrate may be no more than about 40%, no more than about 35%, no more than about 30%, no more than about 25%, no more than about 20%, no more than about 15%, no more than about 10%, or no more than about 5%, where the percentage is taken relative to the average of these concentrations on the surface and in the bulk or center of the substrate.

The distribution of the metal-chelating moiety within the substrate may be relatively uniform, for example, if the metal-chelating moiety is formed as an integral part of the substrate as the substrate is formed. For instance, the metal-chelating moiety may be incorporated within a polymer as a monomer within the polymer, thus resulting in a relatively uniform distribution of the metal-chelating moiety within the polymeric substrate.

In some embodiments, metal ions may be allowed to become distributed within the substrate, e.g., by exposing or the substrate to a fluid containing the metal ions, for example, such that the ions are able to penetrate the substrate via diffusion or other forces (e.g., charge attraction). In some cases, the substrate may be immersed in the fluid. The metal ions may become distributed within the substrate uniformly or non-uniformly, e.g., depending on the length of exposure. For instance, if the metal-chelating moiety is distributed relatively uniformly within the substrate, then metal ions attracted to the metal-chelating moiety may likewise become relatively uniformly within the substrate.

As mentioned, in accordance with certain aspects, particles or other substrates such as those described herein may be useful for purifying proteins and other analytes. For example, suitable tagged proteins or other analytes may be attracted to the metal ions contained within the substrate. As discussed below, in some cases, this association is not permanent and may be reversed, e.g., upon exposure to a suitable competitor to cause the protein or other analyte to dissociate from the substrate.

In one set of embodiments, a protein or other analyte may be tagged with a tag that is attracted to metal ions. One non-limiting examples of a suitable metal-binding tag is a histidine tag. A typical histidine tag includes at least one, and sometimes more, histidine moieties, typically at an end of the analyte (for instance, the N- and/or C-terminus of a protein), which are attracted to certain metal ions such as nickel. In some cases, the tag may include 2, 3, 4, 5, 6, 7, 8, 9, 10, or more histidine moieties, which may be consecutively located.

The tag may be added to the protein or other analyte using any suitable technique. For instance, the tag may be incorporated within the protein when the protein is synthesized (e.g., incorporated into part of the genetic code used to synthesize the protein, for example, to be expressed by a microorganism), or the tag may be added to the protein afterwards, e.g., by a chemical reaction.

Accordingly, by exposing particles or other substrates such as those described herein to a sample suspected of containing a tagged analyte, such as a protein, at least some of the analyte may become immobilized within or with respect to the substrate. The analyte may become bound within the substrate substantially evenly within the substrate, or the distribution may be non-uniform, for example, relatively more concentrated towards the surface of the substrate.

This can be used, for example, to purify a tagged protein or other analyte, to collect or concentrate the protein or other analyte, or the like. For example, a sample suspected of containing an analyte such as a protein may be exposed to a substrate as discussed herein, and the analyte may be allowed to bind to the substrate, e.g., to metal ions contained within the substrate via the metal-binding tags. After exposure, the sample may be removed from the substrate. For instance, the sample may be washed or flushed from the substrate, or if particles are used, the sample may be removed via centrifugation, filtration, or other techniques. In some cases, additional cleaning or rinsing steps may be performed.

The substrate containing the immobilized analyte may then be treated to separate the analyte from the substrate. For instance the substrate may be exposed to a competitor which competes with the immobilized analytes for the metal ions, for example, through competitive inhibition, or through reaction with the metal ions. By increasing the concentration of the competitor to suitable levels, the immobilized analyte may be dissociated, at least partially, from the substrate, e.g., into solution where the analyte may be collected or used for other applications. Non-limiting examples of competitors for metal ions include histidine (as individual amino acids, and/or as free histidine tags), imidazole, or the like.

In another aspect, the present invention is directed to a kit including one or more of the compositions previously discussed. A “kit,” as used herein, typically defines a package or an assembly including one or more of the compositions of the invention, and/or other compositions associated with the invention, for example, as previously described. Each of the compositions of the kit, if present, may be provided in liquid form (e.g., in solution), or in solid form (e.g., a dried powder). In certain cases, some of the compositions may be constitutable or otherwise processable (e.g., to an active form), for example, by the addition of a suitable solvent or other species, which may or may not be provided with the kit. Examples of other compositions that may be associated with the invention include, but are not limited to, solvents, surfactants, diluents, salts, buffers, emulsifiers, chelating agents, fillers, antioxidants, binding agents, bulking agents, preservatives, drying agents, antimicrobials, needles, syringes, packaging materials, tubes, bottles, flasks, beakers, dishes, frits, filters, rings, clamps, wraps, patches, containers, tapes, adhesives, and the like, for example, for using, administering, modifying, assembling, storing, packaging, preparing, mixing, diluting, and/or preserving the compositions components for a particular use, for example, to a sample and/or a subject.

A kit of the invention may, in some cases, include instructions in any form that are provided in connection with the compositions of the invention in such a manner that one of ordinary skill in the art would recognize that the instructions are to be associated with the compositions of the invention. For instance, the instructions may include instructions for the use, modification, mixing, diluting, preserving, administering, assembly, storage, packaging, and/or preparation of the compositions and/or other compositions associated with the kit. In some cases, the instructions may also include instructions for the use of the compositions, for example, for a particular use, e.g., to a sample. The instructions may be provided in any form recognizable by one of ordinary skill in the art as a suitable vehicle for containing such instructions, for example, written or published, verbal, audible (e.g., telephonic), digital, optical, visual (e.g., videotape, DVD, etc.) or electronic communications (including Internet or web-based communications), provided in any manner.

In some embodiments, the present invention is directed to methods of promoting one or more embodiments of the invention as discussed herein. As used herein, “promoted” includes all methods of doing business including, but not limited to, methods of selling, advertising, assigning, licensing, contracting, instructing, educating, researching, importing, exporting, negotiating, financing, loaning, trading, vending, reselling, distributing, repairing, replacing, insuring, suing, patenting, or the like that are associated with the systems, devices, apparatuses, articles, methods, compositions, kits, etc. of the invention as discussed herein. Methods of promotion can be performed by any party including, but not limited to, personal parties, businesses (public or private), partnerships, corporations, trusts, contractual or sub-contractual agencies, educational institutions such as colleges and universities, research institutions, hospitals or other clinical institutions, governmental agencies, etc. Promotional activities may include communications of any form (e.g., written, oral, and/or electronic communications, such as, but not limited to, e-mail, telephonic, Internet, Web-based, etc.) that are clearly associated with the invention.

In one set of embodiments, the method of promotion may involve one or more instructions. As used herein, “instructions” can define a component of instructional utility (e.g., directions, guides, warnings, labels, notes, FAQs or “frequently asked questions,” etc.), and typically involve written instructions on or associated with the invention and/or with the packaging of the invention. Instructions can also include instructional communications in any form (e.g., oral, electronic, audible, digital, optical, visual, etc.), provided in any manner such that a user will clearly recognize that the instructions are to be associated with the invention, e.g., as discussed herein.

U.S. Provisional Patent Application Ser. No. 61/445,942, filed Feb. 23, 2011, entitled “Particles and Other Substrates Useful in Protein Purification and Other Applications,” by Mizrahi, et al., is incorporated herein by reference in its entirety.

The following examples are intended to illustrate certain embodiments of the present invention, but do not exemplify the full scope of the invention.

### Example 1

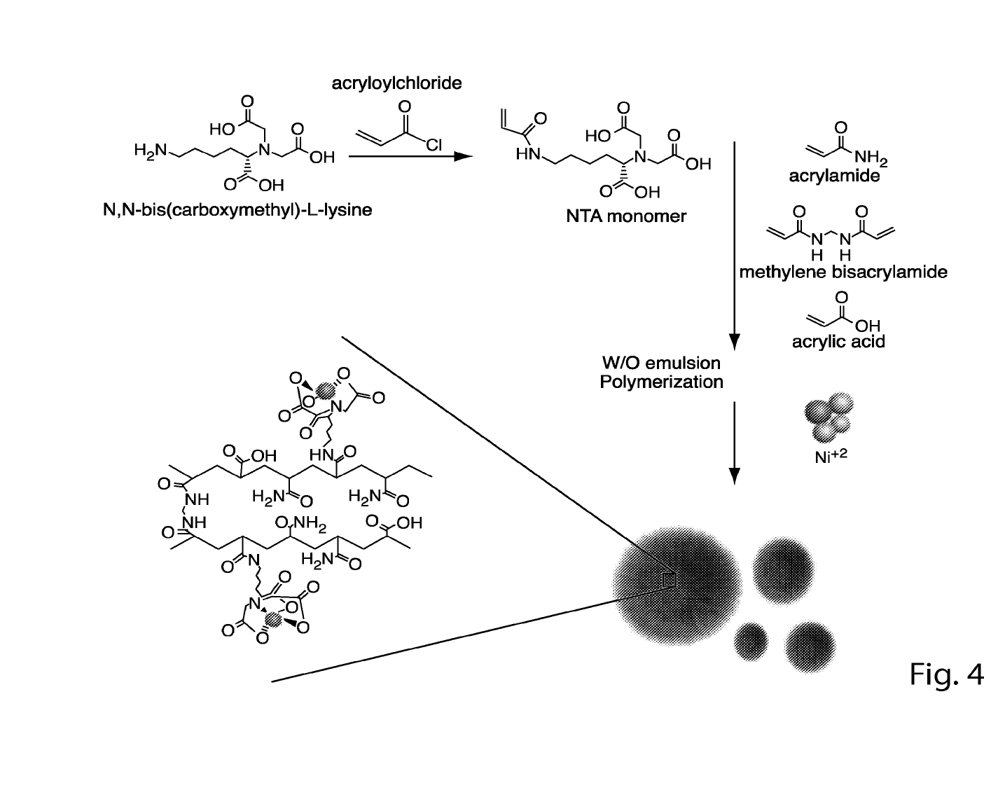

This example demonstrates that the efficiency of His-tagged protein purification can be improved by enhancing the penetration of proteins into the matrix while presenting a high density of metal ions. A synthetic scheme is presented in FIG. 4 to produce particles where a metal-chelating moiety, nitrilotriacetic acid (NTA), is distributed throughout the entire matrix. The NTA monomer is 2,20-(5-acrylamido-1-carboxypentylazanediyl)diacetic acid, which allows the NTA to be incorporated within a polymer. Although NTA is used in this example, other metal-chelating moieties may be used in other embodiments. The absence of a separate coating step may, in some cases, reduce the clogging of matrix pores during synthesis.

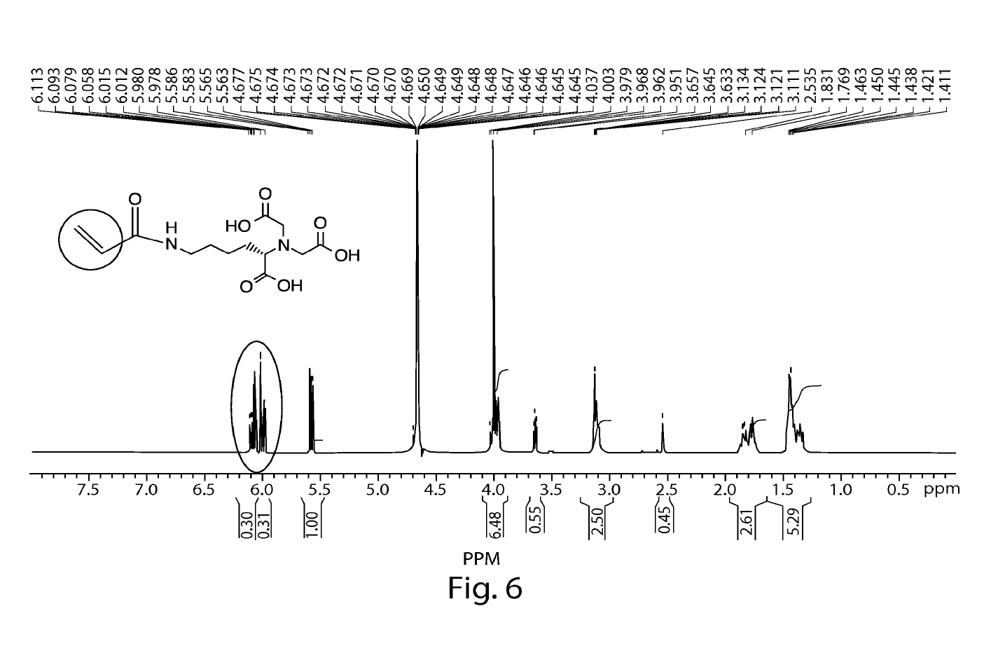

In this example, the NTA monomer was formed by reacting N,N-bis(carboxymethyl)-L-lysine in 0.4 M NaOH solution with acryloylchloride in toluene (upper reaction in FIG. 4). The solvent was evaporated in vacuo followed by removal of sodium ions. The NTA monomer was obtained by lyophilization; its identity (i.e., 2,20-(5-acrylamido-1-carboxypentylazanediyl)diacetic acid) was confirmed by ¹H NMR (FIG. 6). FIG. 6 shows the ¹H NMR spectra (in D₂O) of the product of reaction between N,N-bis(carboxymethyl)-L-lysine and acryloylchloride. The presence of two new peaks around 6 ppm (circled) for the two protons of the carbon double bond (circled in structure) documented the synthesis of 2,20-(5-acrylamido-1-carboxypentylazanediyl)diacetic acid (NTA-monomer).

15 mol % NTA monomer was dissolved in Tris/HCl buffer along with 66 mol % acrylamide, 2.6 mol % N,N′-methylenebisacrylamide, and 16.4 mol % acrylic acid to impart a negative charge to the eventual particle. A water-in-oil emulsion was produced by dropwise addition of this solution to dodecane with 1% Span 80 (sorbitan monooleate). The emulsion was probe-sonicated and purged with nitrogen. Redox polymerization was initiated by adding 150 microliters aqueous ammonium persulfate solution (0.1 g/mL) and 100 microliters N,N,N′,N′-tetramethylethylendiamine (TEMED), thereby causing polymerization of the monomers into polymeric particles. The particles were precipitated with methanol for 1 hour, isolated by centrifugation and suspended in concentrated 1.5 M aqueous NiSO₄ solution for 12 h to produce Ni²⁺ charged microgel particles. Lastly, the Ni²⁺ charged microgel particles were washed with deionized water to remove the unbound nickel ions. The resultant microgel particles were uniform in size, with an average diameter of 6.5±0.8 micrometers (determined using a Coulter counter).

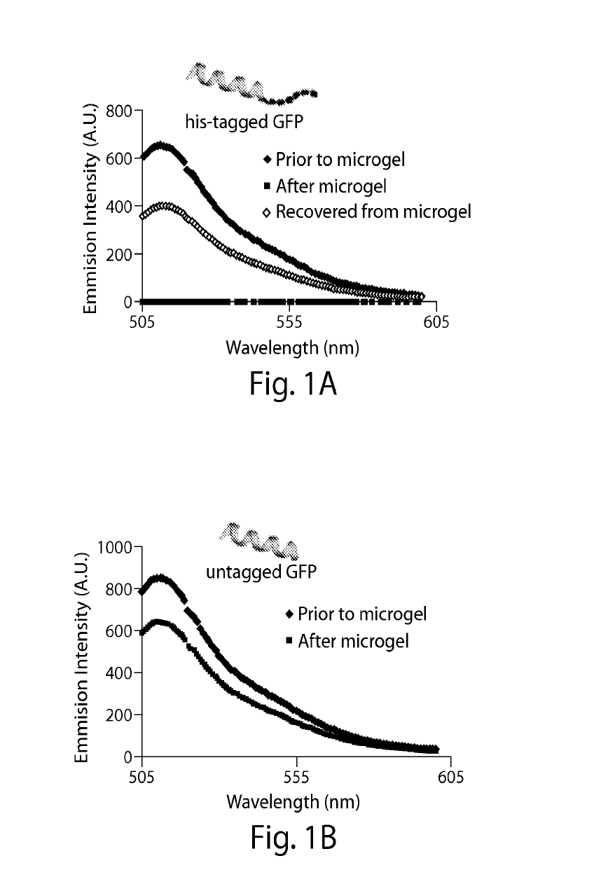

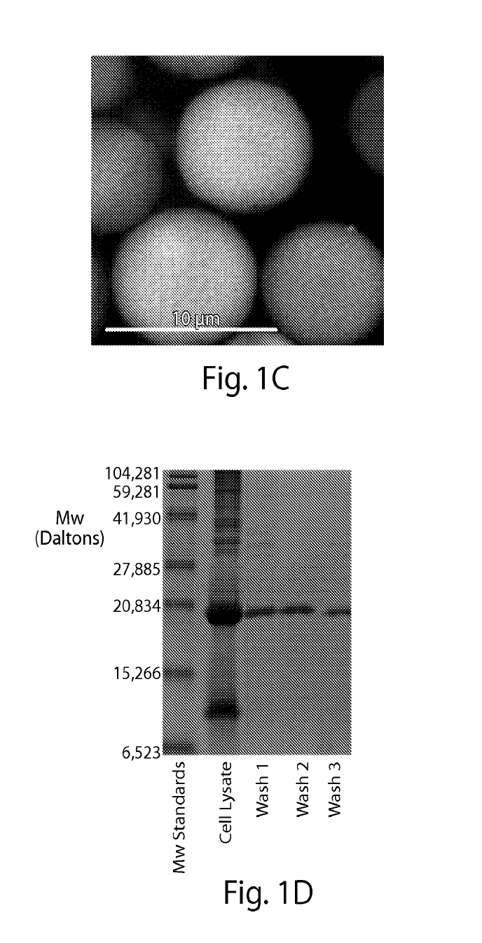

To evaluate the ability of the microgel (formed from the above-described microgel particles) to reversibly and selectively bind His-tagged proteins, 0.2 mg of the microgel were incubated for 20 min at room temperature in 20 microgram/600 microliter solutions of His-tagged GFP (green fluorescent protein) or untagged GFP (which have comparable molecular weights, 29 kDa and 26.8 kDa, respectively). Green fluorescent protein is readily available commercially from a number of different sources. The microgel was separated from the supernatants by centrifugation (1,000 RPM, 2 min) and 600 microliters of 300 mM imidazole solution was added to release the bound protein from the nickel. Imidazole is a competitor of histidine. The binding and the recovery abilities of the microgels were determined from the emission intensities of the media (FIGS. 1A and 1B).

FIG. 1A shows the emission intensities of the fluorescence spectra of His-tag GFP free in solution before (upper curve) and after (lower curve) binding with microgels. The middle curve represents the protein recovered after application of 300 mM imidazole solution. FIG. 1B shows a fluorescence spectra of the emission intensities of untagged GFP in the solutions before (upper curve) and after (lower curve) microgel introduction.

The addition of the microgel containing microgel particles, followed by the removal of the particles, reduced the emission intensity of the His-tagged GFP in free solution to near zero, indicating a very high binding efficiency. In contrast, the concentration of untagged GFP hardly changed, indicating little binding to the microgels in the absence of the histidine tag. Treatment of the His-tagged GFP loaded microgels (FIG. 1C, a fluorescent image of microgel particles loaded with His-tag GFP) with 300 mM imidazole solution released the protein with a recovery efficiency of approximately 65% (determined from FIGS. 1A and 1B by the NIH ImageJ analysis program).

To determine the ability of the microgels to purify His-tagged proteins directly from a cell lysate, in another set of experiments, 1 mg of microgel was introduced into a suspension of recombinant His-tagged ferritin lysed extracts (˜20 kD, from E. coli BL21). The suspension was agitated for 20 min at 4° C., then the microgels were separated by centrifugation (2,000 RPM, 2 min). The particles were washed with deionized water to remove residual lysate, and then washed with 40 mM imidazole solution (wash 1 in FIG. 1D) and with 300 mM solution (washes 2 and 3 in FIG. 1D). The proteins were collected from each step for analysis by SDS-PAGE (sodium dodecyl sulfate polyacrylamide gel electrophoresis) (FIG. 1D) which confirmed the purification of the desired protein. Furthermore, only trace amounts of other proteins were washed off by the 40 mM imidazole solutions, suggesting minimal nonspecific interaction with proteins. In particular, FIG. 1D shows SDS-PAGE of His-tag ferritin isolated from cell lysates with microgels. From left to right: FIG. 1D shows molecular weight standards, cell lysates, proteins washed from microgels with 40 mM imidazole solution (Wash 1), and twice with 300 mM imidazole solution (Washes 2 and 3).

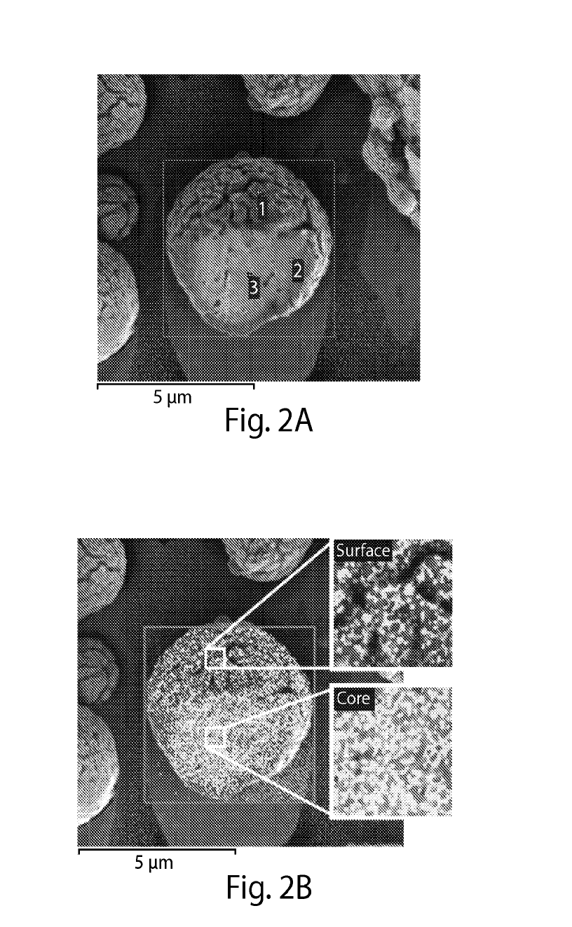

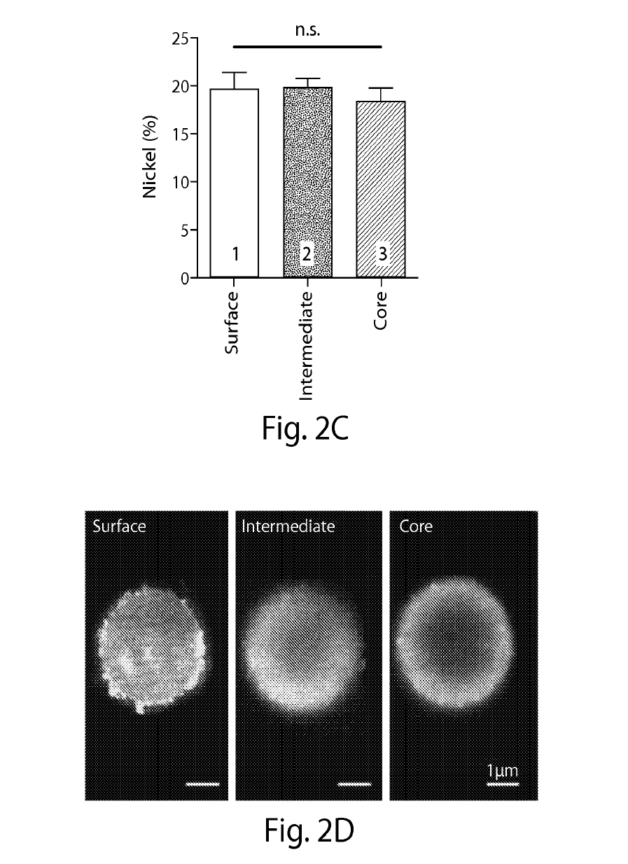

The internal structure and nickel ion density in the microgel were examined by dual-beam microscopy (a combination of a focused ion beam with an electron beam) that allows SEM imaging and local elemental analysis by energy-dispersive X-ray (EDX) of localized cross-sections (FEI Nova 200 Nanolab). The microgel particles exhibited micrometer-scale corrugated features with channels on the outer surface and pores in the body (FIG. 2A). This figure shows SEM micrographs of ion-milled microgel particle displaying the surface and the core. Numbers in FIG. 2A indicate locations analyzed for nickel content at (1) the surface, (2) an intermediate location, and (3) the core.

This structure has been attributed to a decrease in cross-link density from the center toward the periphery of the particles. An EDX map for nickel of the same particle (FIG. 2B) showed nickel throughout the particle. In particular, FIG. 2A shows an SEM/EDX map for nickel in the same particle shown in FIG. 2A. Nickel is indicated by dots. The insets are enlargement of the surface and the core. The pores are in black.

The nickel densities (FIG. 2C) were approximately 20% w/w at three locations starting at the surface then progressing to the center of the microgel (marked in FIG. 2A). The nickel mean density (% w/w) throughout the microgel particle is shown at the locations indicated in FIG. 2A. Data are mean±SD (n=8); n.s.=no statistically significant difference by ANOVA. These results confirmed that this method produced high concentrations of nickel from surface to core.

Confocal laser scanning microscopy (FIG. 2D) of the microgels incubated in His-tagged GFP solution (6 microgram/250 microliter) for 1 hour showed penetration of the His-tagged protein to a depth of approximately half the radius of the particle (i.e. roughly 82.5% of the sphere volume). This figure is a fluorescent confocal micrographs of a microgel incubated in His-tagged GFP solution, showing the surface and cross sections at depths of 1.5 micrometers (intermediate) and 3 micrometers (core). The excitation wavelength was 488 nm.

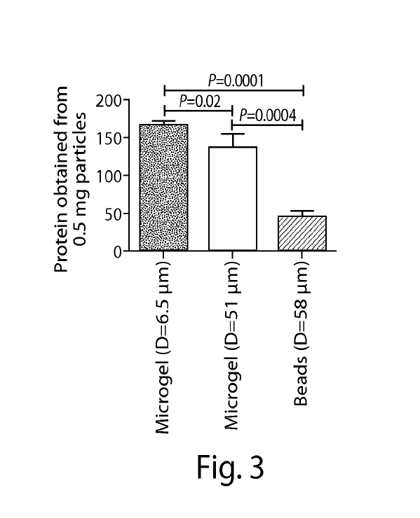

The capacity of 0.5 mg of microgels to bind proteins was quantified by incubating the microgel particles in 200 microliters of cell lysate, then measuring the eluted proteins with a Bradford Coomassie brilliant blue assay (FIG. 3). This figure shows the efficiency of microgels of different sizes and of commercially available beads in purifying His-tagged ferritin from cell lysate. Data are means±SD (n=8).

By way of comparison, the same mass of commercially available beads (Ni-NTA Agarose, Qiagen Inc., Chatsworth, Calif.) bound around 3.7 times less protein. To address the potential contribution of differences in particle size between the commercial beads and microgels (58±15.6 micrometers vs. 6.5±0.8 micrometers, respectively), microgel particles of equivalent size were synthesized (51.1±21 micrometers). With these particles, the protein yields per unit mass of the microgels were 3 times higher than of the commercially available beads, indicating that regardless of size, the microgel may be more efficient than conventional beads. In addition, these results show that the size of the particles may not be a dominant factor in determining protein binding, and that protein binding occurs substantially evenly throughout these particles.

Measurements of the particle specific surface area, pore volume, and average pore width were performed using the Brunauer-Emmett-Teller (BET) nitrogen adsorption method with ASAP 2020 accelerated surface area and porosimetry analyzer. The accessible surface area (Table 1) of the commercial beads was almost 3 times lower than of the microgel particles of the same size. The specific surface area of the smaller microgels was 26% larger than for the larger ones. The pore volumes were ten times larger in the microgels than in the commercial beads, and the pore widths were three times larger.

TABLE 1

| | | | Pore | |
|---|---|---|---|---|
| | Average size | Surface Area | volume | Average pore |
| Sample | (micrometers) | (m²/g) | (cm³/g) | width (nm) |
| Microgel #1 | 6.5 ± 0.8 | 12.95 ± 0.04 | 0.0286 | 8.83 |
| Microgel #2 | 51.1 ± 21 | 10.28 ± 1.28 | 0.0301 | 9.98 |
| Commercial | 58 ± 15.6 | 3.52 ± 0.29 | 0.0029 | 3.27 |

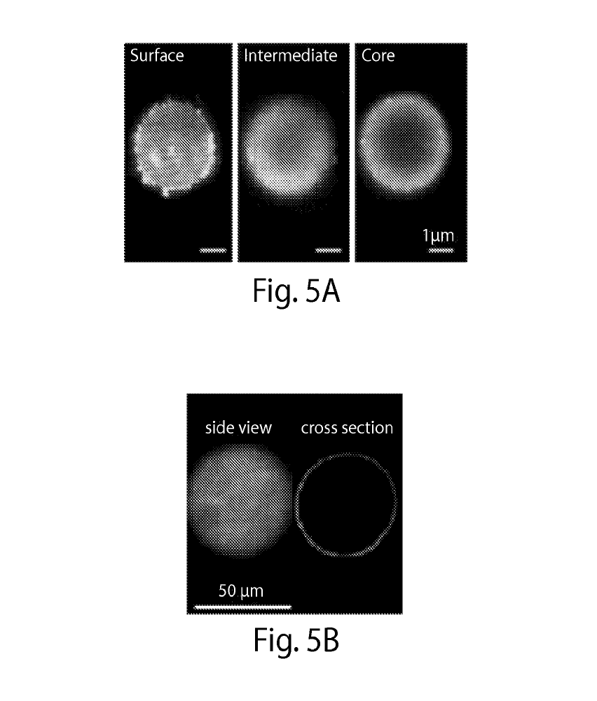

The enhanced performance of the microgels was potentially due to contributions from two factors. One was the relatively high Ni content in the microgels (see FIG. 2C) compared to 3.2±1.8% in the commercial particles (p<0.0002, compared to the 6.5 micrometer and 51 micrometer microgel particles). The other factor was the potential Ni-bearing surface area available for protein binding. The microgels allowed much deeper penetration of proteins within the particles (FIG. 5). In particular, FIG. 5 shows confocal micrographs of microgel particles (FIG. 5A) and of commercially available beads (Ni-NTA Agarose, Qiagen Inc., Chatsworth, Calif.) (FIG. 5B), showing a surface view and cross sections. The images were taken after incubation for 1 hour in His-tagged GFP solution. The excitation wavelength was 488 nm.

The particle size could also affect the surface available, in that it determines the surface area to volume ratio of the particles. In fact, the 22% higher protein binding in the smaller microgels compared to the larger microgels (p<0.02) was very similar to the difference in specific surface area between them (26%). The slightly higher protein binding by the smaller microgels was not the primary reason for the emphasis on them; they were easier to manufacture, had less tendency to aggregate, and could be easier to dispense.

In summary, these experiments show the successful synthesis of protein-binding microgels from NTAs and other monomers. The particles were produced by simple synthetic steps amenable to large-scale production, which may reduce the high costs associated with many protein purification systems. The versatile acrylic backbone allows easy tuning of particle properties to modify performance as desired. When loaded with Ni²⁺, the microgels offered an efficient alternative to current methods of enriching, immobilizing, and purifying proteins and possibly other biomolecules. This new system has a high nickel density, and is easily penetrated by proteins in solutions. Particle size and degree of crosslinking may also play a role in formulation performance.

### Example 2

This example illustrates various techniques useful in Example 1. Reaction solvents were of analytical grade and were used as received from Omnisolv. Acryloylchloride, NaOH, urea, N,N-bis(carboxymethyl)-L-lysine, acrylamide, N,N′-Methylenebisacrylamide, acrylic acid, imidazole, ammonium persulfate, Tris/HCl, N,N,N′,N′-tetramethylethylendiamine (TEMED), dodecane, Dowex 50WX8, and span 80 were purchased from Sigma-Aldrich. Recombinant His-tagged Enhanced Green Fluorescent Protein (≧97% purity, Ex./Em.=488 nm/507 nm) was purchased from Cell Sciences® (Canton, Mass.). Recombinant Green Fluorescent Protein (≧95% purity, Ex./Em.=395 nm/507 nm) was purchased from Abcam® (Canton, Mass.). All data collected are presented as mean±standard deviation of at least four samples. Student's t-test was used to compare data sets. p values<0.05 were considered to reflect statistical significance.

The modification of N,N-bis(carboxymethyl)-L-lysine was documented by ¹H-NMR spectra using a Varian Mercury 500 MHz spectrometer at 25° C. in D₂O. Photoluminescent spectra were collected on a Tecan Infinite M200 micro plate reader (Tecan Austria, Austria) with an excitation wavelength of 488 nm (for His-tagged Green Fluorescent Protein) and 395 nm (for untagged Green Fluorescent Protein). Fluorescent images of the microgel loaded with His-tagged Green Fluorescent Protein were obtained by using fluorescence microscopy (Carl Zeiss, Inc., model HAL 100, Germany). Confocal microscopy was performed on a PerkinElmer Ultraview Spinning Disk system (PerkinElmer, USA) mounted on a Zeiss Axiovert 200m (Carl Zeiss Microimaging, Germany).

Fabrication of microgel particles in a water-in-oil emulsion: 530 microliters of acryloylchloride were dissolved in 25 mL toluene and added dropwise to an ice-cooled 0.4 M NaOH solution of N,N-bis(carboxymethyl)-L-lysine (1.6 g in 50 mL). The solution was stirred overnight followed by the evaporation of toluene by rotary evaporation. Sodium ions were removed with Dowex® 50WX8 (Sigma). Dowex® was washed several times with DDW (doubly deionized water) until pH 7 was achieved. Then, lyophilization was carried out, which resulted in thick oil. 0.23 g of 2,20-(5-acrylamido-1-carboxypentylazanediyl)diacetic acid, 0.227 g acrylamide, 20 mg N,N′-methylenebisacrylamide, 55 microliters of acrylic acid and ammonium persulfate solution (0.1 g/mL, 150 microliters) were dissolved in 8 mL 50 mM Tris/HCl, pH 8.5, under nitrogen. A concentrated W/O (water/oil) emulsion was formed by dropwise addition of the monomers solution into a continuous oil phase (dodecane plus 1% Span 80). The emulsion was probe-sonicated (Vibra Cell Sonicator, Sonics & Materials, Danbury, Conn.) in a 100 mL flask for 30 seconds (15 cycles of 2 seconds, with 2 seconds of no sonication in between) using a probe set at 40% power and purged with nitrogen to remove residual oxygen. Redox polymerization of the concentrated W/O emulsion was initiated by adding 100 microliters of TEMED, and the reaction was allowed to proceed for 1 hour. The particles were precipitated with methanol, isolated by centrifugation (1,500 RPM for 3 min) and re-suspended in NiSO₄ hexahydrate aqueous solution (1.5 M) for 12 h. The Ni²⁺-charged microgel particles were washed and centrifuged 6 times to separate the microgel particles from unbound nickel ions. Microgels of larger size were fabricated by homogenizing similar monomeric solutions as mentioned above at 2,000 RPM.

Milling and nickel ions density and imaging: A Dual Beam from FEI, model Nova Nanolab 200 (XT Nova Nanolab, Hillsboro, Oreg., USA) was used. The cutting/milling technique uses a dense beam of Ga⁺ ions to mill deep trenches in the area of interest. The source of the electron beam is a field emission gun with accelerating voltages of between 5 kV and 30 kV. SEM (scanning electron microscopy) images of the site-specific sample have been taken using field emission SEM operating at 200 eV to 30 keV.

Surface area measurements were carried out using the BET nitrogen adsorption method with an ASAP 2010 apparatus (Micromeritics, Japan), after pre-treating the samples overnight under vacuum at room temperature. For the calculation of BET specific surface area, relative pressures in the range of 0.05 to 0.2 were used.

Protein purification procedure: Isopropyl-beta-D-thiogalactopyranoside induced E. coli bacterial cultures expressing His-tagged human ferritin were pelleted and frozen at −80° C. overnight. The cells were lysed using a 1× concentration of Sigma Cellytic B Cell Lysis Reagent, 100 microliters of 100 mg/mL lysozyme, and ˜250 units of Sigma Benzonase nuclease. The lysis solution was spun at 16,000 RPM for 20 minutes, re-suspended in 20 mM Tris (base), 6 M urea, and spun again at 16,000 RPM for 20 minutes. The lysate protein solution was exposed to the particulate affinity matrices for about 20 min, centrifuged (1,000 RPM, 2 min) and washed with a 20 mM Tris, 20 mM NaCl, pH 8.1, and with a 50 mM Tris pH 8.1, 50 mM NaCl, 40 mM imidazole solution. The histidine-tagged protein solution was eluted from each matrix using a 50 mM Tris pH 8.1, 50 mM NaCl, 300 mM imidazole solution.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A composition, comprising:
a particle formed from polymer, the particle comprising a metal-chelating moiety, wherein the metal-chelating moiety is distributed substantially evenly throughout the particle.

2. The composition of claim 1, wherein the particle consists essentially of the polymer.

3. The composition of claim 1, wherein the metal-chelating moiety within the polymer is formed from at least nitrilotriacetic acid.

4. The composition of claim 1, wherein the polymer is a copolymer formed from a plurality of monomers.

5. The composition of claim 4, wherein one monomer of the plurality of monomers forming the polymer is nitrilotriacetic acid.

6. The composition of claim 4, wherein one monomer of the plurality of monomers forming the polymer is acrylamide.

7. The composition of claim 4, wherein one monomer of the plurality of monomers forming the polymer is N,N′-methylenebisacrylamide.

8. The composition of claim 4, wherein one monomer of the plurality of monomers forming the polymer is acrylic acid.

9. The composition of claim 1, wherein the particle further contains metal ions.

10. The composition of claim 9, wherein at least some of the metal ions are divalent metal ions.

11. The composition of claim 9, wherein at least some of the metal ions are Ni²⁺ ions.

12. The composition of claim 9, wherein the metal ions are distributed substantially evenly throughout the particle.

13-15. (canceled)

16. The composition of claim 1, wherein the particle has an average diameter of less than about 60 micrometers.

17. The composition of claim 1, wherein the particle has an average surface area of at least about 5 m²/g.

18-19. (canceled)

20. The composition of claim 1, wherein the particle has an average pore volume of at least about 0.005 cm³/g.

21-22. (canceled)

23. The composition of claim 1, wherein the particle has an average pore width of at least about 5 nm.

24-25. (canceled)

26. A method, comprising exposing the composition of claim 1 to a sample comprising a protein.

27. (canceled)

28. A method, comprising exposing the composition of claim 1 to a sample comprising a histidine-tagged analyte.

29. The method of claim 28, wherein the analyte is a protein.

30. A method of releasing a histidine-tagged analyte from a particle, the method comprising:
exposing a particle suspected of being exposed to a histidine-labeled analyte to a histidine competitor, wherein the particle contains a metal ion distributed substantially evenly throughout the particle.

31. The method of claim 30, wherein the histidine competitor comprises histidine.

32. The method of claim 30, wherein the histidine competitor comprises imidazole.

33-35. (canceled)

36. A method, comprising:
exposing a solution comprising a metal-chelating moiety and acrylamide to a liquid that is immiscible with the solution to form droplets of the solution contained within the liquid;
polymerizing the metal-chelating moiety with the acrylamide to form polymeric particles; and
separating the polymeric particles from the liquid.
